(19) United States
(12) Patent Application Publication
(10) Pub. No.: US 2010/0112627 A1
(43) Pub. Date: May 6, 2010

(54) System and Method for Displaying Three-Dimensional Object Scattergrams

(75) Inventors: Patricio J. VIDAL, Miami, FL (US); Haiping Jia, Miami, FL (US)

Correspondence Address: STERNE KESSLER GOLDSTEIN & FOX, P.L.L.C., 1100 NEW YORK AVENUE, N.W., WASHINGTON, DC, 20005, US

(73) Assignee: Beckman Coulter, Inc., Fullerton, CA (US)

(21) Appl. No.: 12/609,843

(22) Filed: Oct. 30, 2009

Related U.S. Application Data
(60) Provisional application No. 61/193,195, filed on Nov. 4, 2008.

(51) Int. Cl. C12Q 1/02; C12M 1/34; G01N 21/00

(52) U.S. Cl. 435/29; 435/288.7

(57) ABSTRACT

System and method for displaying three-dimensional object scattergrams of particles are provided. In one embodiment, at least two parameters associated with at least one particle in a biological sample are detected and stored as data. An initial two-dimensional scattergram of the data is created with the two dimensions corresponding to the two parameters, and each data point corresponding to a particle in the biological sample. A data point in the initial two-dimensional scattergram is categorized into a population corresponding to a particle population. A density value of the data point is evaluated. Color data for the data point is calculated based on the evaluated density value and the categorized population. A three-dimensional location is generated based on the location in the initial two-dimensional scattergram and a property of the data point. A geometric shape centered at the generated three-dimensional location is displayed using the calculated color data.

### CROSS-REFERENCE TO RELATED APPLICATIONS

The present application claims the benefit of U.S. Provisional Application No. 61/193,195, filed Nov. 4, 2008, which is incorporated by reference herein in its entirety.

### FIELD OF THE INVENTION

Embodiments of the present invention generally relate to systems and methods of particle analysis. More specifically, embodiments of the present invention relate to systems and methods for processing and displaying cellular analysis data.

### BACKGROUND

Particle analyzers, such as, flow cytometers and hematology analyzers, measure physical properties of particles in a biological sample. Exemplary hematology analyzers are available from a number of companies including Beckman Coulter Inc., Sysmex Corp., Abbott Laboratories Inc., Siemens AG, and Shenzhen Mindray Bio-Medical Electronics Co., Ltd. Exemplary flow cytometers are available from a number of companies including Beckman Coulter Inc. and Becton, Dickinson and Company. Exemplary physical property measurements performed by particle analyzers include electro-optical measurements.

The measurements of the physical properties of the biological particles are stored as particle analysis data. Each measured physical property corresponds to a parameter in the particle analysis data. Particle analyzers often provide users with the display of two-dimensional (2D) scattergrams or histograms representing data measuring two parameters.

Each data point in the 2D scattergram corresponds to a particle event. Color can be used to differentiate particle populations in scattergrams displayed to a user. FIG. 1 illustrates a conventional scattergram of two blood cell measurements where different colors (shown in different shades of gray in FIG. 1) are used to represent different populations. However, conventional scattergrams fail to show density information when the data points start overlapping each other. Overlapping data points are common in scattergrams that display as many as several thousands of events or more.

Two-dimensional histograms can provide density information about data points in the data. A two-dimensional histogram contains a set of two-dimensional bins. Each bin accumulates particle events appearing at the location of the bin. The accumulated value represents the particle density at the location. In an exemplary two-dimensional histogram, bitmaps are used to represent the density ranges of the two-dimensional bins. For example, in a 256×256 histogram containing 64×64 bins, the density ranges can be represented as bitmaps of 4×4 pixels. Each bitmap represents a density range.

In two-dimensional histograms, the number of colored pixels in the bitmaps can correspond to the density ranges. For example, a high number of pixels in a bitmap are colored for a high density range. A relatively low number of pixels in the bitmaps are colored for low density ranges. Different colors can also be used for different particle populations in the histograms.

FIG. 2 illustrates, in grayscale, a conventional two-dimensional color histogram 210 generated based on blood cell analysis data. Each cell population is identified by color-related bitmaps (shown in different shades of gray in FIG. 2). Each bitmap has a color pattern corresponding to the density of the respective cells. Each cell population can be represented by a family of 10 color patterns. Different color patterns contain different numbers of colored pixels. Bitmaps having color patterns with more colored pixels indicate higher densities in the respective population. For example, in histogram 210, retic cells are represented by 4×4 bitmaps with color patterns formed by blue, cyan, or gray pixel colors (illustrated here in different shades of gray). Mature red blood cells are represented by bitmaps in red, dark red, or gray colors. Platelets are represented by bitmaps in green or cyan colors.

Image 220, shown in grayscale, is an enlarged version of region 212 in histogram 210. Each color square in image 220 represents a pixel (e.g., pixel 222). Each block of 4×4 color squares represents a bitmap (e.g., bitmaps 224, 226, 228). Different blocks have different color patterns based on the respective cell densities. Image 220 represents a region containing retic cells. Bitmaps 224 and 226 have the same color pattern shown here in different shades of gray. Each of bitmaps 224 and 226 contain 7 colored pixels, 4 in blue (shown as a first shade of gray), 1 in cyan (shown as a second shade of gray), and 2 in gray (shown as a third shade of gray). Bitmap 228 has a different color pattern containing 9 colored pixels with 2 in gray (the third shade of gray) and 7 in blue (the first shade of gray). Accordingly, bitmap 228 corresponds to a higher density of retic cells than bitmaps 224 and 226.

By visually inspecting these two-dimensional histograms, users can identify and compare the density information of the different blood cell populations for patient diagnosis and result validation. However, drawbacks exist for conventional histogram displays. Histograms reduce the resolution of the data because of the data binning, which causes the perception of individual events to be lost.

Because conventional two-dimensional scattergrams and histograms only contain information about two parameters, particle populations that share similar values over the two parameters can overlap with each other. It is difficult to differentiate such populations in the conventional two-dimensional scattergrams or histograms.

### BRIEF SUMMARY OF THE INVENTION

Embodiments of the present invention provide systems and methods for displaying three-dimensional (3D) object scattergrams of particle analysis data. These 3D object scattergrams are an enhancement compared to existing 2D scattergrams.

In one embodiment, a method includes steps of preparing a biological sample for analysis, passing biological particles from the biological sample through a measuring region of a particle analyzer, and interrogating each particle passing through the measuring region to detect at least two parameters. The parameters are detected by one or more detectors and stored as data. An initial two-dimensional scattergram of the data is created with a first dimension corresponding to a first parameter and a second dimension corresponding to a second parameter. Each data point corresponds to a particle in the biological sample. Each data point in the initial two-dimensional scattergram is categorized into a particle population. A density value for each data point is evaluated. Color data for each data point is calculated based on the evaluated density value. According to a feature of the present invention, a three-dimensional location is generated based on the location in the initial two-dimensional scattergram and a property of each data point. A geometric shape centered at the generated three-dimensional location is then rendered using the calculated color data for each respective data point.

Another embodiment of the present invention is directed to a system for displaying a three-dimensional object scattergram for particle analysis. The system includes a particle analyzer, a 3D object scattergram generator, and a renderer. The particle analyzer provides data for particle analysis by processing a biological sample. The particle analyzer includes a preparation system and a transducer module. The transducer module has one or more interrogation sources and one or more detectors. The interrogation sources provide electro-optical interrogations of the particles. The detectors detect a plurality of parameters associated with the particles. The 3D object scattergram generator generates three-dimensional scattergram data based on the provided data. The renderer renders geometric shapes based on the generated three-dimensional scattergram data.

The 3D object scattergram generator includes an initial scattergram creator, a population analyzer, a density evaluator, a color calculator, and a coordinate generator. The initial scattergram creator creates an initial two-dimensional scattergram based on the data provided by the measuring device. The population analyzer categorizes each data point in the initial two-dimensional scattergram into a respective population. Each population corresponds to a respective particle population in the sample. The density evaluator determines a respective density value for each data point. The density value corresponds to the number of data points in the respective population within a specified distance from the data point in the initial two-dimensional scattergram. The color calculator calculates color data for each data point based on the respective population and density value. The coordinate generator determines a coordinate value in a third dimension for each data point based on a property of the data point. A three-dimensional location is generated for each data point based on the respective location in the initial two-dimensional scattergram and the respective coordinate value in the third dimension.

One feature of the present invention is to provide three-dimensional object scattergrams of particle analysis data. The three-dimensional object scattergrams can clearly show different particle populations and their density information. The results can help a user understand and analyze the data even containing a large number of data points. The three-dimensional object scattergrams also incorporate information in a third dimension, which can display particle populations blocked by others in conventional two-dimensional scattergrams or histograms.

Further embodiments, features, and advantages of the present invention, as well as the structure and operation of the various embodiments of the present invention, are described in detail below with reference to the accompanying drawings.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is an image of a conventional two-dimensional scattergram for a blood sample.

FIG. 2 is an image of a conventional two-dimensional histogram generated by a Beckman Coulter hematology analyzer based on a two-dimensional scattergram.

FIG. 3 is a diagram of an exemplary system for displaying two-dimensional scattergrams according to an embodiment of the invention.

FIG. 4 is a diagram of an exemplary particle analyzer for providing data for display according to an embodiment of the invention.

FIG. 5 is a flowchart of an exemplary procedure for displaying two-dimensional scattergrams according to an embodiment of the invention.

FIG. 6 is an image of an exemplary 3D object scattergram, according to an embodiment of the invention.

FIG. 7A an image of an exemplary 3D object scattergram using conductivity for z values, according to an embodiment of the invention.

FIG. 7B is an image of an exemplary 3D object scattergram using dynamically assigned z values, according to an embodiment of the invention.

FIG. 8 is a diagram of an exemplary computer system for displaying 3D object scattergrams according to an embodiment of the invention.

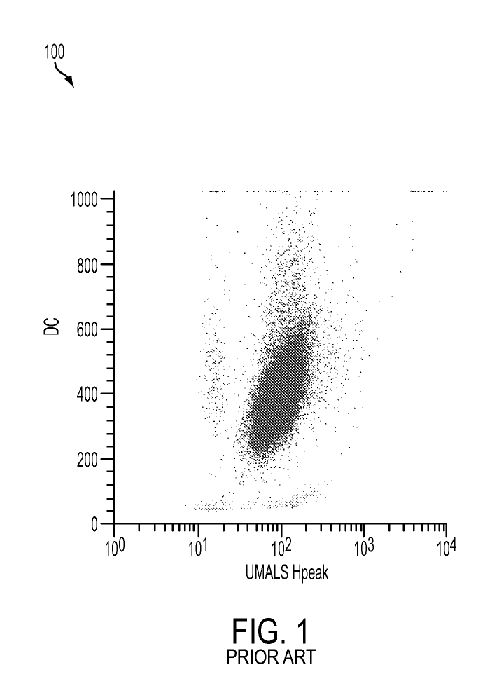

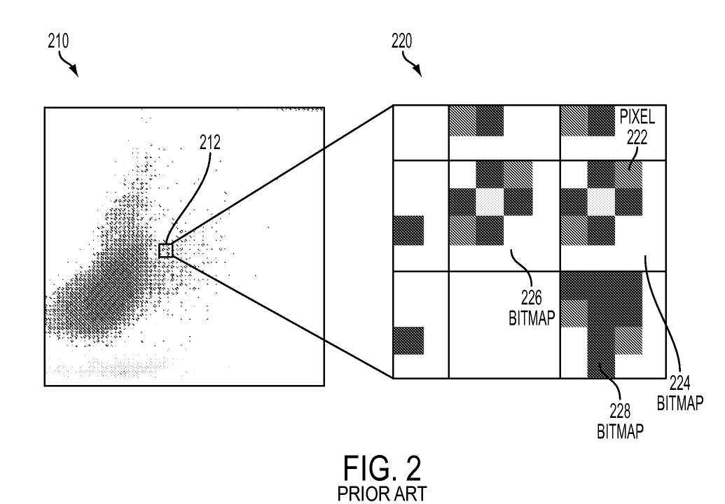

The present invention is described with reference to the accompanying drawings. The drawing in which an element first appears is typically indicated by the leftmost digit in the corresponding reference number.

### DETAILED DESCRIPTION OF EMBODIMENTS OF THE INVENTION

Embodiments of the present invention are described with respect to particle analyzers. One feature is to display 3D object scattergrams of particle analysis data that clearly show the different particle populations and their density information. The resulting display can help a user understand and analyze the data even for a large number of data points.

Embodiments of the present invention provide methods and systems for displaying particle analysis data of a biological sample using a 3D object scattergram. In the 3D object scattergram, each particle population in the data is represented by a base color. Each data point is represented as a geometric shape (e.g. three-dimensional sphere). Local density information is represented by changing a color parameter (e.g. brightness).

In one embodiment for displaying whole blood sample data, three-dimensional spheres are used as the geometric shape to represent the data points in the three-dimensional object scattergram. Data points are categorized into cell populations. The base color of a sphere is assigned based on the respective cell population. For example, red is assigned to mature red blood cells, blue is assigned to retic cells, and green is assigned to platelets, etc. This example is for illustrative purpose and not intended to limit the present invention. Other geometric shapes, colors, and color assignments can be used.

A color parameter such as brightness can be used to represent the density information for the data points. In one embodiment, the brightness of the color assigned to a data point is proportional to the data point density in its neighborhood area. Higher densities result in brighter colors. Lower densities are represented by darker colors. In this way, color still identifies a particle population while brightness of the color shows density information in a three-dimensional object scattergram.

In one embodiment, each data point in particle analysis data is processed for the 3D object scattergram. In alternative embodiments, only a portion of the data points are processed. For example, in one embodiment, only data points with density values above a pre-selected threshold are processed. In another example, only data points in selected populations are processed.

### System Overview

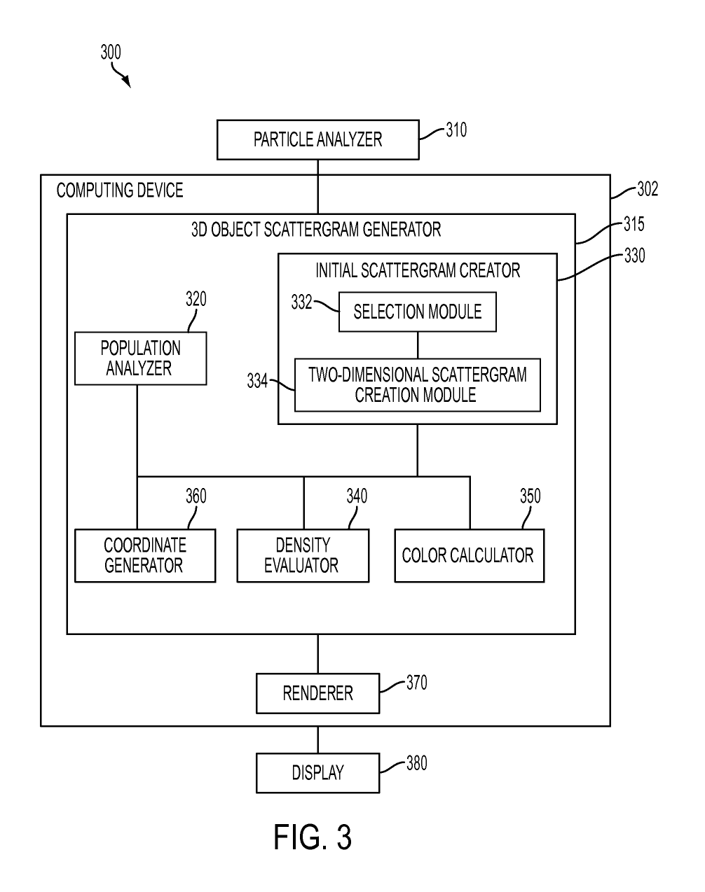

FIG. 3 is a diagram of an exemplary system 300 for displaying three-dimensional object scattergrams according to an embodiment of the invention. System 300 includes a particle analyzer 310, a computing device 302, and a display 380. Computing device 302 includes a three-dimensional object scattergram generator 315 and a renderer 370. In another embodiment (not shown), display 380 can be included in computing device 302.

Particle analyzer 310 provides data for particle analysis by processing biological samples of particles. For example, in one embodiment, the particles are blood cells. In alternative embodiments, the particles can be other types of biological particles such as DNA fragments. These examples are illustrative and not intended to limit the present invention.

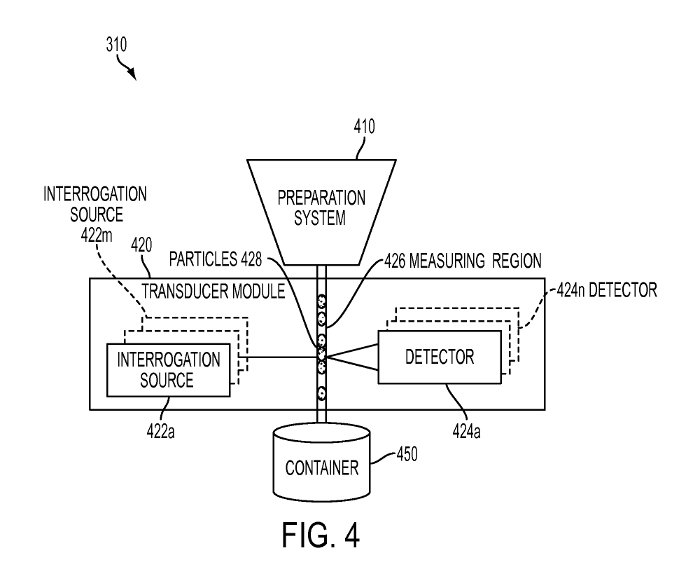

FIG. 4 is a schematic diagram of particle analyzer 310 according to an embodiment of the present invention. Particle analyzer 310 includes a preparation system 410, a transducer module 420, a measuring region 426, and a container 450.

Preparation system 410 prepares biological samples containing particles for analysis.

Transducer module 420 provides data corresponding to the particles to be analyzed. In one embodiment, transducer module 420 includes multiple interrogation sources 422a-m, multiple detectors 424a-n, and a measuring region 426. Preparation system 410 passes the prepared biological particles 428 from the biological sample through measuring region 426.

Interrogation sources 422a-m provide electro-optical interrogations of particles 428 so that one or more parameters associated with the interrogated particles can be detected by detectors 424a-n respectively. In one embodiment, for example, interrogation sources 422a-m can include one or more laser and/or electrical sources. In alternative embodiments, other types and/or numbers of interrogation sources can be used. Detectors 424a-n detect parameters associated with the interrogated particles in the samples. In one embodiment, detectors 424a-n include one or more photodiode sensors. Detectors 424a-n can also include a DC receiver. In alternative embodiments, other types of detectors can be used.

In one embodiment, the parameters are derived from electro-optical measurements which include DC (direct current), RF (radio frequency), one or more types of light scatter (at one or more angles), fluorescence, and axial light loss as described, for example, in U.S. Pat. No. 5,125,737 which is hereby incorporated in its entirety by reference, and known in the art. These examples of parameters are illustrative and not intended to limit the present invention.

In one embodiment, particle analyzer 310 measures the biological particles contained in a flowing stream. In another embodiment, particle analyzer 310 measures the biological particles in a microtiter plate. Particle analyzer 310 can be any analyzer that can provide particle analysis data in a multidimensional feature space.

Three-dimensional (3D) object scattergram generator 315 generates a three-dimensional object scattergram for data received from particle analyzer 310. The data includes detected parameters associated with the biological sample. Three-dimensional object scattergram generator 315 includes a population analyzer 320, an initial scattergram creator 330, a density evaluator 340, a color calculator 350, and a coordinate generator 360.

Initial scattergram creator 330 creates an initial scattergram based on the data received from particle analyzer 310. Initial scattergram creator 330 includes a selection module 332 and a two-dimensional scattergram creation module 334. Selection module 332 selects data corresponding to two detected parameters from the data received from particle analyzer 310. Two-dimensional scattergram creation module creates a two-dimensional scattergram using the selected data corresponding to the parameters. The two dimensions of the two-dimensional scattergram correspond to the two parameters, respectively.

Population analyzer 320 categorizes the data received from particle analyzer 310 into the particle populations. Data points in the initial scattergram are associated with the categorized particle populations. Various techniques can be used to categorize the particle populations. For example, in one embodiment, population analyzer 320 creates one-dimensional histograms and/or two-dimensional histograms over all the detected parameters and their combinations. By analyzing the created histograms, the data can be categorized into particle populations. In another embodiment, statistics of the data can be used to categorize the particle populations. These examples are illustrative and not intended to limit the present invention.

Density evaluator 340 evaluates local density information for a data point in the initial two-dimensional scattergram. The local density information can be represented by or based upon the number of data points in a neighborhood area of the data point in a same particle population. For example, in one embodiment, population analyzer 320 receives blood sample data from particle analyzer 310. Population analyzer 320 categorizes the data into mature red blood cell, retic cell, and platelet populations. Data points in the initial two-dimensional scattergram are associated with respective populations accordingly. The local density for a data point associated with the platelet population is the number of platelet data points in its neighborhood. Similarly, the local density for a retic cell data point is the number of retic cell data points in its neighborhood. The local density for a matured red blood cell data point is the number of mature red blood cell data points in its neighborhood. This example is illustrative and not intended to limit the present invention. For instance, the local density for a data point is not limited to the number of data points in the neighborhood area of a population and instead could be a function of the number of data points (such as a weighted function) depending upon a particular application.

Color calculator 350 assigns color data to a data point based on its population and density information. In one embodiment, color calculator 350 assigns different base colors to different populations. Color calculator 350 then calculates the color brightness for a data point in a population based on its density information.

Coordinate generator 360 generates a three-dimensional location for a data point. The three-dimensional location contains the two-dimensional coordinate of the data point in the initial scattergram and a coordinate value in a third dimension. The third dimension can correspond to a property of the data point. For example, the property can be the population the data point is associated with, a value with respect to another parameter detected by particle analyzer 320, etc. In one embodiment, coordinate generator 360 determines a different range of coordinate values in the third dimension for each population. For each data point in a population, coordinate generator 360 assigns a value in the respective range as its coordinate value in the third dimension. In another embodiment, the value in the third dimension is determined based on another detected parameter. For example, in one embodiment the first and second dimensions of the scattergram correspond to DC and light scatter, and the third dimension corresponds to opacity. In this way, populations overlapping in the initial scattergram can be separated because they can have different coordinate values in the third dimension.

Renderer 370 renders a geometric shape using the determined color data and the generated three-dimensional location. The rendered geometric shape is centered at the generated three-dimensional location. In one embodiment, a three-dimensional sphere is used as the geometric shape. The rendered results can be output to display 380 for display.

Display 380 can be any type of display that can be coupled with computing device 302. For example, display 380 can be a CRT monitor, an LCD display, a touchscreen display, etc. These examples are illustrative and not intended to limit the present invention. In another embodiment, the rendered geometric shapes are stored on a storage device (not shown) such as a hard disk, a flash drive, a floppy disk, memory, etc.

In one embodiment, three-dimensional object scattergram generator 315, its components, and renderer 370 can be implemented in software, firmware, hardware or any combination thereof in computing device 302. Example computing devices, include, but are not limited to, a computer, workstation, distributed computing system, embedded system, stand-alone electronic device, networked device, rack server, a device having at least one processor and memory, or other type of computer system.

### Generating Three-Dimensional Object Scattergrams

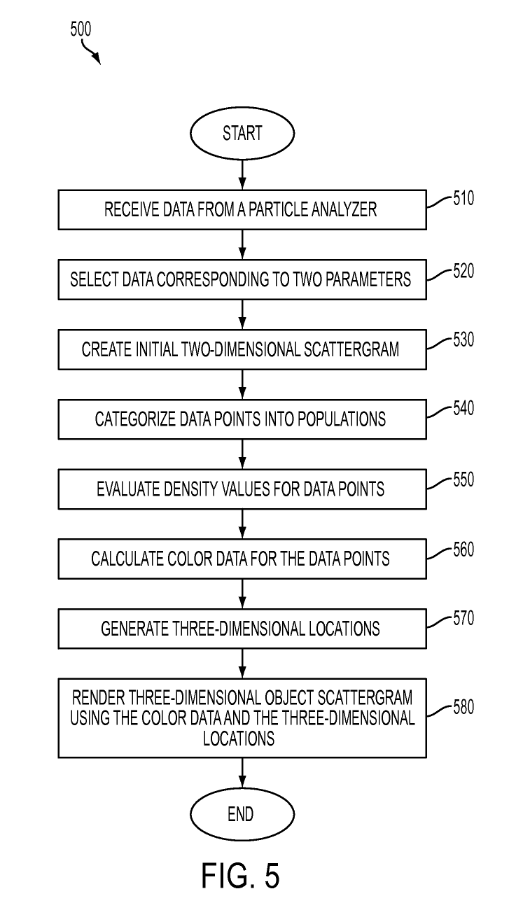

FIG. 5 is a flowchart of an exemplary procedure 500 for generating and displaying 3D object scattergrams (steps 510-580) according to an embodiment of the invention. Although procedure 500 will be described for brevity with reference to system 300 shown in FIG. 3, procedure 500 can be used in other systems for displaying scattergrams.

In step 510, three-dimensional object scattergram generator 315 receives data from particle analyzer 310. The data can include detected parameters associated with a biological sample of particles. For example, in one embodiment, the parameters are derived from electro-optical measurements, which include DC (direct current), RF (radio frequency), light scatter (at one or more angles), fluorescence, side scatter light, and axial light loss as described in U.S. Pat. No. 5,125,737 (incorporated in its entirety herein by reference) and as well-known in the art. Parameters based on the detected parameters, such as opacity, which is a function of DC and RF can be used. These examples of parameters are illustrative and not intended to limit the present invention.

In step 520, selection module 332 selects data corresponding to two detected parameters from the received data. For example, in one embodiment, data corresponding to light scatter and volume are selected. This example is illustrative and not intended to limit the present invention.

In step 530, two-dimensional scattergram creation module 334 then creates an initial two-dimensional scattergram based on the selected data. Each data point in the initial scattergram corresponds to a particle event in the received data.

In step 540, population analyzer 320 categorizes the received data into populations. These populations correspond to the particle populations in the biological sample. Data points in the initial scattergram are associated with the categorized populations. In one embodiment with hematology data of blood samples, the populations include red blood cells, white blood cells, retic cells, platelets, etc. Various techniques can be used to determine the particle populations. For example, in one embodiment, population analyzer 320 creates one-dimensional histograms and/or two-dimensional histograms over all the detected parameters and their combinations. By analyzing the created histograms, particle populations in the data can be determined. In one embodiment, segmentation techniques, such as Watershed transformation, are used to segment the two-dimensional histogram into regions. The regions contain data point clusters corresponding to particle populations. In another embodiment, statistics of the data can be used to determine the particle populations. These examples are illustrative and not intended to limit the present invention.

In step 550, density evaluator 340 evaluates a density value for the data point. Density value is calculated based on the number of nearby data points in the population containing the data point. In one embodiment, a two-dimensional histogram is generated for each particle population. Each two-dimensional histogram provides density information about the respective particle population. The two-dimensional histogram accumulates the number of data points in the population into a 64×64 scale. The two-dimensional scattergram is divided into 64×64 regions that match the two-dimensional histograms. Each region in the two-dimensional scattergram corresponds to an accumulated number in each of the histograms. For each data point in a region, its density value is assigned based on the accumulated number in the two-dimensional histogram for the corresponding population. In another embodiment, the density of a data point is calculated by counting data points in its corresponding population within a pre-determined distance to the data point in the initial scattergram.

### Using Color to Show Population Density

In step 560, color calculator 350 calculates color data for the data point based on its density value. In one embodiment, each population determined by population analyzer 320 is assigned a different base color. For example, in one embodiment, red is assigned to the population of red blood cells, and green is assigned to the population of platelets. This example is illustrative and not intended to limit the present invention. The color data for the data point can be calculated based on the base color for the corresponding population and the density value of the data point. The density value can correspond to the value of a color parameter. In one embodiment, the color parameter is the color brightness.

In an exemplary embodiment, groups of data points are created for each population based on their density values. Each group has the same base color assigned to the corresponding population but with different brightness. The color brightness for each group is proportional to the density range of the group. In this way, data points in a group with higher densities have brighter color, and data points in another group with lower densities have darker color.

For example, in one embodiment, a particle population (such as red blood cells) is divided into five groups using the thresholds of {10, 20, 100, 190}. For a given data point P(x, y) in the population in the initial two-dimensional scattergram, if P(x, y) has a density value d(x,y)≦10, P(x, y) is added to the first group G1. If 10<d(x,y)≦20, P(x, y) is added to the second group G2. If 20<d(x,y)≦100, P(x, y) is added to the third group G3. If 100<d(x,y)≦190, P(x, y) is added to the fourth group G4. Finally, if d(x,y)>190, P(x, y) is added to the fifth group G5.

The first group, G1, is assigned the base color of the population. For groups G2 to G5, their colors are calculated as follows in terms of red, green, and blue component values:

Color(Gi+1).Red=White.Red*0.2+Color(Gi).Red*0.8

Color(Gi+1).Green=White.Green*0.2+Color(Gi).Green*0.8

Color(Gi+1).Blue=White.Blue*0.2+Color(Gi).Blue*0.8

Other particle populations (such as white blood cell) can also be divided into groups and assigned varying brightness based on corresponding density values.

### Generating Three-Dimensional Locations

One feature of the present invention is representing data points using geometric shapes at three-dimensional locations. This feature can discriminate data points that would overlap in a conventional scattergram. In step 570, coordinate generator 360 generates a three-dimensional location for the data point. In one embodiment, the three-dimensional location for the data point is generated based on the two-dimensional location (e.g., (x,y)) of the data point in the initial scattergram and a coordinate value in a third dimension (e.g., z). Several techniques can be used to determine the coordinate value in the third dimension for the data point. For example, another detected parameter value of the data point can be used as the z value, or the z value can be determined by population information associated with the data point.

### Using Another Parameter as the z Value:

In an exemplary embodiment, light scatter and volume are used for the x-y dimensions, and conductivity is used for the z values.

### Dynamic z Value Assignment:

In another embodiment, the z values can be divided into ranges and dynamically assigned to different populations. In such ways, smaller populations can appear in front of bigger populations to make them visible in a display. For example, a range of z values (e.g., 0 to 49) can be assigned to white blood cells, another range (e.g., 50 to 99) can be assigned to platelets, another range (e.g., 100 to 149) can be assigned to retic cells, and another range (e.g., 150 to 199) can be assigned to red blood cells. These examples are illustrative and not intended to limit the present invention. For instance, other ranges of values can be used.

Because the received data is considered random, the dynamically assigned z values can be sequential within each range without removing the “randomness” of the data. For example, in one embodiment, the first 50 data points of white blood cells are assigned a z value of 0 to 49 respectively. Similarly, the next data points 51 to 100 of white blood cells are also assigned a z value of 0 to 49 respectively. The first 50 data points of platelets are assigned a z value of 50 to 99 respectively. The next data points 51 to 100 of platelets are also assigned a z value of 50 to 99 respectively. The first 50 data points of retic cells are assigned a z value of 100 to 149 respectively. The next data points 51 to 100 of retic cells are also assigned a z value of 100 to 149 respectively. The first 50 data points of red blood cells are assigned a z value of 150 to 199 respectively. The next data points 51 to 100 of red blood cells are also assigned a z value of 150 to 199 respectively. This is repeated for data points of other populations. Such z value assignment can ensure consistency when generating three-dimensional object scattergrams. Unlike random z value assignment that can produce different z values every time the data is processed, this population-based z value assignment will generate the same three-dimensional object scattergram for the same data. This dynamic z value assignment is illustrative and not intended to limit the present invention. Other z value assignments can be used depending upon a particular application, including but not limited to, randomly assigning z values to data points within a population.

### Rendering and Displaying Data Points

In step 580, renderer 370 renders a 3D object scattergram using the color data and three-dimensional location information. Renderer 370 can select a geometric shape to represent the data points. In one embodiment, a three-dimensional sphere is used as the geometric shape. The scattergram is rendered as a collection of three-dimensional spheres representing the data points using the calculated color data, where the spheres are centered at the generated three-dimensional locations.

In one embodiment, to improve the system performance, only the visible geometric shapes are rendered and displayed. In general, only data points with the smallest z values are visible when compared with other data points in the same (x,y) coordinates, since they are on top of such data points. Therefore, among all of the data points have same (x,y) coordinates, only the data points with smallest z values are rendered and displayed.

Renderer 370 can further use any type of engine or library to carry out rendering as described herein, for example, renderer 370 can use an OPENGL renderer available from SGI, inc., DIRECT3D available from Microsoft, Inc., or other types of renderers.

### EXAMPLES

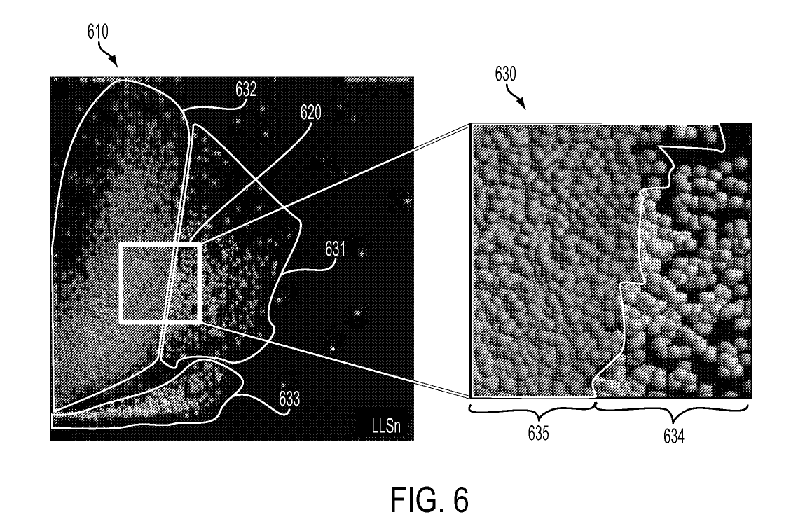

FIG. 6 is a grayscale representation image 610 of an exemplary 3D object scattergram originally generated in color according to an embodiment of the invention. The two dimensions in image 610 are volume (DC) and a transformation of light scatter. Region 620 in image 610 is enlarged to image 630 to show more details. In the exemplary color 3D object scattergram, blue spheres represent retic cells, red spheres represent mature red blood cells, and green spheres represent platelets. The color brightness of the spheres represent density information of the data points represented by the spheres. Spheres having the same base color but different brightness represent variation in density. The brighter spheres indicate higher density in their populations and in their neighborhoods. For example, lighter blue spheres represent retic cells with higher density than retic cells represented by darker blue spheres. In the corresponding grayscale representation image 610, the retic cells, the mature red blood cells, and the platelets, are shown in regions 631, 632, and 633, respectively, in different shades of gray. Density information within each cell population is shown as changes in grayscale brightness, for example, as shown in regions 635 and 634. For example, in the retic cell population shown in region 634, the lighter spheres represent retic cells with higher density than retic cells represented by the darker spheres. In the original color scattergram, these spheres were light blue and dark blue, respectively, as previously mentioned. Similarly, in the mature red blood cell population shown in region 635, the lighter spheres represent mature red blood cells with higher density than the mature red blood cells represented by the darker spheres. In the original color scattergram, these spheres were light red and dark red, respectively.

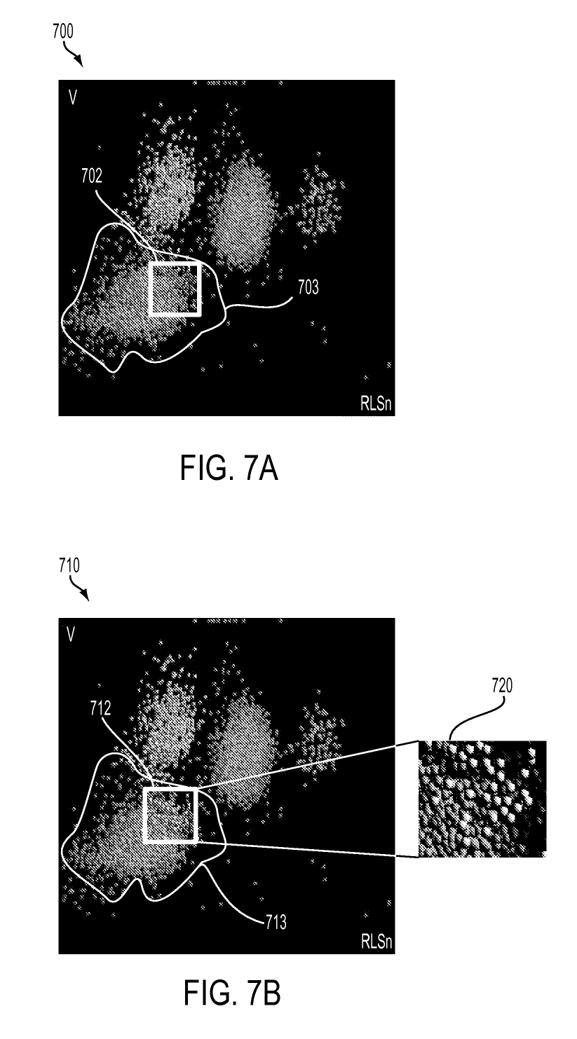

FIG. 7A is a grayscale representation image 700 of an exemplary color 3D object scattergram which uses conductivity for z values, but does not dynamically assign z values based on populations. In the exemplary color 3D object scattergram, white spheres are used to represent basophils and blue spheres are used to represent lymphocytes. However, most white spheres representing basophils are covered by blue data points representing lymphocytes. This is because basophils generally have higher conductivity values than lymphocytes and therefore the corresponding white spheres have larger z values than the blue spheres corresponding to lymphocytes. This effect is illustrated in the corresponding grayscale image 700. Region 703 contains the populations of lymphocytes as well as basophils. In image 700, most of the white spheres (basophils) are covered by data points of a darker gray shade (lymphocytes) in region 702. Therefore, the spheres corresponding to lymphocytes are displayed on top of the spheres corresponding to basophils in region 702 of image 700.

FIG. 7B is a grayscale representation image 710 of an exemplary color scattergram in which conductivity is used for z values and z values are dynamically assigned based on populations. In the color scattergram with dynamically assigned z values, white spheres representing basophils are assigned smaller z values while blue spheres representing lymphocytes are assigned larger z values, thereby displaying the white spheres on top of the blue spheres. Therefore, in the corresponding grayscale representation image 710, the white spheres (basophils) are displayed on top of the darker gray spheres (lymphocytes) in region 712. Region 712, which is part of region 713 that contains basophil and lymphocyte populations, in grayscale image 710 is enlarged in 720 to show more details. In 720, the white spheres (basophils) are clearly visible over the darker gray spheres (lymphocytes).

In this way, particle population and density information can be clearly displayed. Particle populations blocked by others in conventional two-dimensional scattergrams or histograms can also be easily identified in the 3D object scattergram. The 3D object scattergram can help users better understand and analyze the data.

### Exemplary Computer Systems

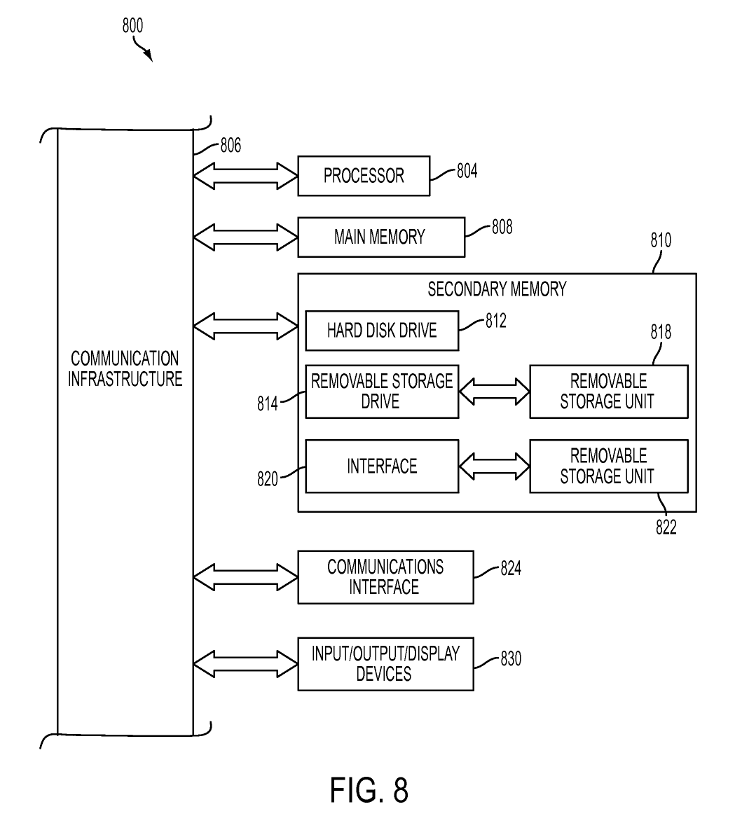

Embodiments of the present invention can be implemented using hardware, firmware, software or a combination thereof and can be implemented in a computer system or other processing system. In an embodiment, a computer program product can execute on a computing device capable of carrying out the functionality described herein. An example of a computing device, computer system 800, is shown in FIG. 8. Computer system 800 includes one or more processors, such as processor 804. Processor 804 is connected to a communication infrastructure 806 (such as a bus).

Computer system 800 also includes a main memory 808, preferably random access memory (RAM), and can also include a secondary memory 810. Secondary memory 810 can include, for example, a hard disk drive 812 and/or a removable storage drive 814, representing a floppy disk drive, an optical disk drive, etc. Removable storage drive 814 reads from and/or writes to a removable storage unit 818 in a well-known manner. Removable storage unit 818, represents a floppy disk, optical disk, memory card, etc. which is read by and written to by removable storage drive 814. As will be appreciated, the removable storage unit 818 includes a computer usable storage medium having stored therein computer software and/or data.

In alternative embodiments, secondary memory 810 can include other similar means, such as a removable storage unit 822 and an interface 820, for allowing computer programs or other instructions to be loaded into computer system 800. Examples of such can include a program cartridge and cartridge interface (such as that found in video game devices), a removable memory chip (such as an EPROM, or PROM) and associated socket, and other removable storage units and interfaces.

Computer system 800 can also include a communication interface 824. Communication interface 824 enables computer 800 to communicate with external and/or remote devices. Examples of communications interface 824 can include a modem, a network interface (such as an Ethernet card), a communications port, a PCMCIA slot and card, etc. Computer system 800 receives data and/or computer program products via communication network 824. Software and data can be transferred via communications interface 824.

Computer programs (also called computer control logic) are stored in main memory 808 and/or secondary memory 810. Computer programs can also be received via communications interface 824. Such computer programs, when executed, enable computer system 800 to perform the features of the present invention as discussed herein. In particular, the computer programs, when executed, enable processor 804 to perform the features of embodiments of the present invention. Accordingly, such computer programs represent controllers of computer system 800.

In an embodiment where the invention is implemented using software, the software can be stored in a computer program product having a tangible computer-readable storage medium and loaded into computer system 800 using removable storage drive 814, hard disk drive 812 or communications interface 824. The control logic (software), when executed by processor 804, causes processor 804 to perform the functions of embodiments of the invention as described herein.

Computer 800 also includes input/output/display devices 832, such as monitors, keyboards, pointing devices, etc.

While various embodiments of the present invention have been described above, it should be understood that they have been presented by way of example, and not limitation. It will be apparent to persons skilled in the relevant computer arts that various changes in form and detail can be made therein without departing from the spirit and scope of the invention. Furthermore, it should be appreciated that the detailed description of the present invention provided herein, and not the summary and abstract sections, is intended to be used to interpret the claims. The summary and abstract sections can set forth one or more but not all exemplary embodiments of the present invention as contemplated by the inventors. Embodiments can work with software, hardware, and operating system implementations other than those described herein. Any software, hardware, and operating system implementations suitable for performing the functions described herein can be used. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalence.

What is claimed is:

1. A method for displaying a three-dimensional object scattergram for particle analysis, comprising:
(a) preparing a biological sample for analysis;
(b) passing biological particles from the biological sample through a measuring region of a particle analyzer;
(c) interrogating each particle passing through the measuring region with at least two parameters;
(d) detecting the at least two parameters with one or more detectors;
(e) storing the detected at least two parameters as data;
(f) creating an initial two-dimensional scattergram of the data, wherein a first dimension of the initial two-dimensional scattergram corresponds to a first parameter, a second dimension of the initial two-dimensional scattergram corresponds to a second parameter, and each data point in the initial two-dimensional scattergram corresponds to a particle in the biological sample;
(g) categorizing a data point in the initial two-dimensional scattergram into a population corresponding to a particle population;
(h) evaluating a density value for the data point;
(i) calculating color data for the data point based on the evaluated density value and the categorized population;
(j) generating a three-dimensional location based on the location in the initial two-dimensional scattergram and a property of the data point; and
(k) rendering a geometric shape centered at the generated three-dimensional location using the calculated color data.

2. The method of claim 1, wherein the biological sample is a blood sample and step (b) comprises passing cells from the blood sample.

3. The method of claim 1, wherein step (h) comprises:
determining a number of data points in the population within a specified distance from the data point in the initial two-dimensional scattergram; and
calculating the density value based on determined number.

4. The method of claim 1, wherein step (i) comprises:
assigning initial color data with the population of the data point; and
adjusting the color data based on the evaluated density value.

5. The method of claim 1, wherein step (c) further comprises interrogating each particle passing through the measuring region with a third parameter.

6. The method of claim 5, wherein step (j) comprises:
determining a coordinate value in a third dimension to the data point based on the property of the data point; and
generating the three-dimensional location based on the location in the initial two-dimensional scattergram and the assigned coordinate value.

7. The method of claim 6, wherein determining the coordinate value in the third dimension comprises determining the coordinate value in the third dimension based on the third parameter.

8. The method of claim 7, wherein step (d) comprises detecting at least two parameters selected from the following group of parameters: direct current, volume, radiofrequency, opacity, one or more types of light scatter, axial light loss, and fluorescence.

9. The method of claim 6, wherein determining the coordinate value in the third dimension comprises determining the coordinate value based on the population of the data point.

10. The method of claim 1, wherein displaying the geometric shape comprises displaying a sphere having a specified radius.

11. The method of claim 1, wherein the biological sample is a blood sample.

12. The method of claim 1, further comprising displaying the rendered geometric shape.

13. The method of claim 1, further comprising storing the rendered geometric shape.

14. A system for displaying a three-dimensional object scattergram for particle analysis, comprising:
a particle analyzer configured to provide data for particle analysis by processing a biological sample, wherein the particle analyzer comprises:
a preparation system for preparing a biological sample containing a plurality of particles for analysis; and
a transducer module having a measuring region, a plurality of interrogation sources to interrogate at least one of the particles passing through the measuring region, and at least one detector to detect a plurality of parameters associated with at least one of the particles;
a three-dimensional object scattergram generator configured to generate three-dimensional scattergram data based on the provided data, wherein the three-dimensional object scattergram generator comprises:
an initial scattergram creator configured to create an initial two-dimensional scattergram based on the data provided by the particle analyzer;
a population analyzer configured to categorize a data point in the initial two-dimensional scattergram into a population corresponding to a particle population in the sample;
a density evaluator configured to determine a density value for the data point based on a number of data points in the population within a specified distance from the data point in the initial two-dimensional scattergram;
a color calculator configured to calculate color data for the data point based on the population and the density value; and
a coordinate generator configured to determine a coordinate value in a third dimension based on a property of the data point and generates a three-dimensional location for the data point based on a location of the data point in the initial two-dimensional scattergram and the coordinate value in the third dimension; and
a renderer configured to render geometric shapes for display based on the generated three-dimensional scattergram data.

15. The system of claim 14, wherein the initial scattergram creator comprises:
a selection module configured to select first data corresponding to a first detected parameter and second data corresponding to a second detected parameter; and
a two-dimensional scattergram creation module configured to create an initial two-dimensional scattergram using data corresponding to the first and second parameters, wherein a first dimension of the initial two-dimensional scattergram corresponds to the first parameter, and a second dimension of the initial two-dimensional scattergram corresponds to the second parameter.

16. The system of claim 14, wherein the color calculator is configured to:
assign an initial color to the data point based on the population of the data point; and
adjust the assigned color based on the density value of the data point.

17. The system of claim 14, wherein the particle analyzer is configured to detect a third parameter associated with the biological sample of the particles.

18. The system of claim 17, wherein the coordinate generator is configured to determine the coordinate value in the third dimension corresponding to the third parameter.

19. The system of claim 14, wherein the coordinate generator is configured to determine the coordinate value in the third dimension corresponding to the population of the data point.

20. The system of claim 14, wherein the three-dimensional scattergram data comprises, for a data point in the initial two-dimensional scattergram, color data and a three-dimensional location.

21. The system of claim 20, wherein the renderer is configured to:
select a sphere as the geometric shape;
determine a size of the sphere; and
display the sphere using the color data and the determined size, wherein the sphere centers at the three-dimensional location.

22. The system of claim 21, wherein the renderer is configured to display a geometric shape centered at the three-dimensional location using the color data, wherein the three-dimensional location has a smallest coordinate value in the third dimension among three-dimensional locations having a same location in the initial two-dimensional scattergram.

23. The system of claim 14, wherein the plurality of parameters comprises at least two parameters selected from the following group of parameters: direct current, volume, radiofrequency, opacity, one or more types of light scatter, axial light loss, and fluorescence.

24. A method for displaying a three-dimensional object scattergram for particle analysis, comprising:
(a) obtaining an initial two-dimensional scattergram based on particle analysis data corresponding to one or more particles, wherein a first dimension of the initial two-dimensional scattergram corresponds to a first parameter measuring a first property of the particles, a second dimension of the initial two-dimensional scattergram corresponds to a second parameter measuring a second property of the particles, and each data point in the initial two-dimensional scattergram corresponds to a particle;
(b) categorizing a data point in the initial two-dimensional scattergram into a population corresponding to a particle population;
(c) evaluating a density value for the data point;
(d) calculating color data for the data point based on the evaluated density value;
(e) generating a three-dimensional location based on the location in the initial two-dimensional scattergram and a property of the data point; and
(f) displaying a geometric shape centered at the generated three-dimensional location using the calculated color data.

25. A three-dimensional object scattergram generator for generating three-dimensional object scattergram data based on particle analysis data corresponding to one or more particles, comprising for use in a computing device:
an initial scattergram creator that creates an initial two-dimensional scattergram based on the particle analysis data, wherein each data point in the initial scattergram corresponds to a particle;
a population analyzer that categorizes a data point in the initial two-dimensional scattergram into a population corresponding to a particle population in the particles;
a density evaluator that determines a density value for the data point based on a number of data points in the population within a specified distance from the data point in the initial two-dimensional scattergram;
a color calculator that calculates color data for the data point based on the population and the density value; and
a coordinate generator that determines a coordinate value in a third dimension based on a property of the data point and generates a three-dimensional location for the data point based on a location of the data point in the initial two-dimensional scattergram and the coordinate value in the third dimension.
